(19) United States
(12) Patent Application Publication
(10) Pub. No.: US 2011/0150836 A1
(43) Pub. Date: Jun. 23, 2011

(54) METHODS OF TREATING HBV AND HCV INFECTION

(75) Inventors: Randall L. HALCOMB, Foster City, CA (US); Sundaramoorthi Swaminathan, Burlingame, CA (US); Daniel B. Tumas, San Carlos, CA (US)

(73) Assignee: Gilead Sciences, Inc., Foster City, CA (US)

(21) Appl. No.: 12/970,213

(22) Filed: Dec. 16, 2010

Related U.S. Application Data
(60) Provisional application No. 61/289,158, filed on Dec. 22, 2009.
(60) Provisional application No. 61/300,340, filed on Feb. 1, 2010.

(51) Int. Cl. A61K 31/522; A61K 31/7068; A61K 31/675; A61K 31/7072; A61K 38/21; A61K 31/573; A61P 31/20; A61K 31/7056; A61P 31/14

(52) U.S. Cl. 424/85.7; 514/263.2; 514/49; 514/81; 514/50; 514/171; 424/85.4; 514/43

(57) ABSTRACT

This application relates to a purine derivative and pharmaceutical compositions which are useful for treating a hepatitis B viral infection or a hepatitis C viral infection.

### CROSS-REFERENCE TO RELATED APPLICATIONS

This application claims priority to U.S. Provisional Application Ser. No. 61/289,158, filed Dec. 22, 2009, and U.S. Provisional Application Ser. No. 61/300,340, filed Feb. 1, 2010. The content of these provisional applications are herein incorporated by reference in their entirety for all purposes.

### FIELD OF THE INVENTION

This application relates to a purine derivative and pharmaceutical compositions which are useful for treating HBV and HCV infections, as well as to methods of making and using the compound.

### BACKGROUND OF THE INVENTION

Hepatitis B Virus (HBV) infects the liver and causes an inflammation commonly referred to as hepatitis. The disease is endemic in certain populations, such as China and other parts of Asia. About a third of the world's population, more than 2 billion people have been infected with the hepatitis B virus. This includes 350 million chronic carriers of the virus.

Hepatitis B virus infection may either be acute, otherwise known as self-limiting, or chronic, otherwise known as long-standing. Persons with self-limiting infection clear the infection spontaneously within weeks to months. Acute infection with hepatitis B virus is associated with acute viral hepatitis, an illness that begins with general ill-health, loss of appetite, nausea, vomiting, body aches, mild fever, dark urine, and then progresses to development of jaundice. In addition, itchy skin has been indicated as a possible symptom of all hepatitis virus types. The illness lasts for a few weeks and then gradually improves in most affected people. A few patients may have more severe liver disease (fulminant hepatic failure), and may die as a result of it. The infection may be entirely asymptomatic and may go unrecognized. Chronic infection with Hepatitis B virus may be either asymptomatic or may be associated with a chronic inflammation of the liver, chronic hepatitis, leading to cirrhosis over a period of several years. This type of infection dramatically increases the incidence of hepatocellular carcinoma or liver cancer. Chronic carriers are encouraged to avoid consuming alcohol as it increases their risk for cirrhosis and liver cancer.

Transmission of hepatitis B virus results from exposure to infectious blood or body fluids containing blood. Possible forms of transmission include (but are not limited to) unprotected sexual contact, blood transfusions, re-use of contaminated needles, and syringes, and vertical transmission from mother to child during childbirth.

Acute hepatitis B infection does not usually require treatment because most adults clear the infection spontaneously. On the other hand, treatment of chronic infection may be necessary to reduce the risk of cirrhosis and liver cancer. For example, chronically infected individuals with persistently elevated serum alanine aminotransferase, a marker of liver damage, and HBV DNA levels are candidates for therapy.

Approved medications for treatment of HBV in the United States include: lamivudine (Epivir®), adefovir (Hepsera®), tenofovir (Viread®), telbivudine (Tyzeka®) and entecavir (Baraclude®) and two immune system modulators interferon alpha-2a and pegylated interferon alpha-2a (Pegasys®). Infants born to mothers known to carry hepatitis B can be treated with antibodies to the hepatitis B virus (hepatitis B immune globulin or HBIg). In addition, there are several available vaccines against HBV infection. The foregoing drugs can stop the HBV virus from replicating, thereby minimizing liver damage such as cirrhosis and liver cancer, but they cannot clear the virus from the body, and they must be administered to a patient over an extended time period to continually suppress viral replication. They may also have undesirable side effects in individual patients.

The hepatitis C virus is an enveloped, single-stranded, positive sense RNA virus in the family Flaviviridae. HCV mainly replicates within hepatocytes in the liver. Circulating HCV particles bind to receptors on the surfaces of hepatocytes and subsequently enter the cells. Once inside the hepatocyte, HCV utilizes the intracellular machinery necessary to accomplish its own replication. Lindenbach, B. Nature 436(7053):932-8 (2005).

The World Health Organization estimates that world-wide 170-200 million people (3% of the world's population) are chronically infected with HCV. Approximately 75% of these individuals are chronically infected with detectable HCV RNA in their plasma. These chronic carriers are at risk of developing cirrhosis and/or liver cancer. In studies with a 7-16 year follow-up, 7-16% of the patients developed cirrhosis, 0.7-1.3% developed hepatocellular carcinoma and 1.3-3.7% died of liver-related disease.

There are currently limited treatment options available for HCV infection, such as the use of interferon α-2 (or its pegylated form) either alone or combined with ribavirin. Sustained response to interferon is only observed in about 40% of the patients and treatment is associated with serious adverse effects.

Accordingly, there is a continuing need for new compositions of matter and methods for treating HBV infection and HCV infection in human beings. In particular, there is a continuing need for compositions of matter and treatments that induce seroconversion with respect to HBV or HCV whereby an infected individual generates an immune response that suppresses HBV or HCV virus replication.

### SUMMARY OF THE INVENTION

The compound used in the practice of the present invention has the following structure and chemical name:

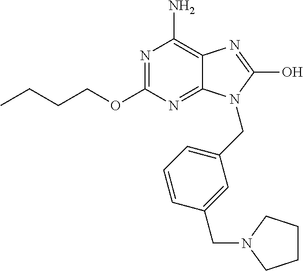

### 6-amino-2-butoxy-9-(3-(pyrrolidin-1-ylmethyl)benzyl)-9H-purin-8-ol

For ease of reference, this compound may be referred to herein as Compound A. It will be understood that in the practice of the present invention reference to Compound A means Compound A or a pharmaceutically acceptable salt thereof.

The present inventors have demonstrated that Compound A induces expression of immunomodulatory cytokines in human peripheral blood mononuclear cells (Example 2), Cynomolgus monkeys (Example 3), mice (Example 4) and healthy woodchucks (Example 5). Moreover, as described in Example 6, the present inventors have demonstrated that Compound A causes seroconversion against Woodchuck Hepatitis Virus (WHV) in chronically infected Eastern Woodchucks (Marmota monax) which is an art-recognized model system for HBV infection in human beings (see, e.g., Tennant, B. C., Animal models of hepatitis B virus infection, Clin. Liver Dis. 3:241-266 (1999); Menne, S., and P. J. Cote, The woodchuck as an animal model for pathogenesis and therapy of chronic hepatitis B virus infection, World J. Gastroenterol. 13:104-124 (2007); and Korba B E, et al., Treatment of chronic WHV infection in the Eastern woodchuck (M. monax) with nucleoside analogues is predictive of therapy for chronic hepatitis B virus infection in man, Hepatology, 31: 1165-1175 (2000)).

After seroconversion, the amount of WHV virus DNA in the blood of the infected woodchuck is reduced, and in some animals is no longer detectable in the blood, and the animal's immune system is capable of suppressing replication of WHV virus particles.

While not wishing to be bound by theory, the inventors currently believe that Compound A is an agonist of Toll-Like Receptor 7 (TLR-7), and may also agonize other members of the TLR family of receptors which are involved in regulation of various aspects of the mammalian immune response, such as production of antibodies and immunomodulatory cytokines (see, e.g., S. Akira and K. Takeda, Toll-like Receptor Signalling, Nature Reviews (Immunology), 4:499-511 (2004)).

Some viruses, such as the human immunodeficiency virus (HIV), cannot be cleared from an infected human being by an immune response, and so infected individuals must undergo a life-long course of therapy with antiviral drugs that suppress replication of the virus. In contrast, both HBV and HCV can be cleared from infected human beings by an immune response, such as production by the infected person of neutralizing antibodies directed against one or more surface antigens of HBV or HCV. It is noteworthy, however, that even in patients who develop an immune response against HBV or HCV, the immune response is often insufficient to clear HBV or HCV. Further, human beings who are infected with HBV as neonates by an infected mother are tolerized to the HBV and do not develop an immune response against this virus.

Thus, the results described in Example 6 of the present application, which were obtained using woodchucks that were infected with WHV at birth, demonstrate the feasibility of using Compound A for treating HBV and HCV by inducing immune responses against these viruses. In particular, the results described in Example 6 of the present application demonstrate the feasibility of using Compound A to cause seroconversion against HBV and HCV even in patients who are infected at birth and who are otherwise tolerized to these viruses.

Accordingly, in one aspect the present invention provides methods for treating a hepatitis B viral infection or a hepatitis C viral infection wherein each of the methods include the step of administering to a human subject (alternatively referred to as a human being) infected with hepatitis B virus or hepatitis C virus a therapeutically effective amount of Compound A or a pharmaceutically acceptable salt thereof. Typically, the human subject is suffering from a chronic hepatitis B infection or chronic hepatitis C infection, although it is within the scope of the present invention to treat people who are acutely infected with HBV or HCV.

Treatment in accordance with the present invention results in the stimulation of an immune response and a reduction in viral load. Thus, if a human being is infected with HBV, then treatment in accordance with the present invention stimulates an immune response against HBV with a consequent reduction of HBV viral load in the infected person. If a human being is infected with HCV, then treatment in accordance with the present invention stimulates an immune response against HCV with a consequent reduction of HCV viral load in the infected person. Examples of immune responses include production of antibodies (e.g., IgG antibodies) and/or production of cytokines, such as interferons or interleukins, that modulate the activity of the immune system. The immune system response can be a newly induced response, or can be boosting of an existing immune response.

Compound A is administered by any useful route and means, such as by oral or parenteral (e.g., intravenous) administration. Therapeutically effective amounts of Compound A, and frequency and route of dosage are described herein. As described more fully herein, Compound A can be administered with one or more additional therapeutic agent(s) to a human being infected with HBV or HCV. The additional therapeutic agent(s) can be administered to the infected human being at the same time as Compound A, or before or after administration of Compound A.

In other aspects, the present invention provides method(s) for ameliorating a symptom associated with an HBV infection or an HCV infection; methods for reducing the rate of progression of a hepatitis B viral infection, or a hepatitis C viral infection, in a human being; methods for reducing the viral load associated with HBV infection or associated with HCV infection; and methods of inducing or boosting an immune response against Hepatitis B virus, or against Hepatitis C virus, in a human being.

Additionally, the present invention provides for the use of Compound A, or a pharmaceutically acceptable salt thereof, to treat a hepatitis B virus infection, or to treat a hepatitis C virus infection, in a human being. Additionally, the present invention includes the use of Compound A, or a pharmaceutically acceptable salt thereof, to manufacture a medicament for the treatment of a hepatitis B virus infection, or the treatment of a hepatitis C virus infection, in a human being.

The present invention also provides methods for ameliorating at least one symptom of HBV or HCV infection in a human being, wherein each method of this aspect of the invention includes the steps of (a) testing a human subject to determine if the human subject is infected with HCV or HBV, and (b) if the human subject is infected with HBV or HCV then administering to the human subject an amount of Compound A that is sufficient to ameliorate at least one symptom of the HCV or HBV infection. Testing can be done, for example, using the Roche COBAS Amplicor® HCV Monitor kit, or the Roche COBAS Amplicor® HBV Monitor kit.

### BRIEF DESCRIPTION OF THE DRAWINGS

The following detailed description will be more fully understood in view of the accompanying drawings in which:

FIG. 1 shows serum WHV DNA concentration plotted against time (weeks) for Woodchucks chronically infected with WHV that were treated with either Compound A or with placebo. The abbreviation “vge/ml” stands for viral genome equivalent per milliliter.

FIG. 2 shows anti Woodchuck Hepatitis Virus Surface Antigen antibody concentration plotted against time (weeks) for Woodchucks chronically infected with WHV that were treated with either Compound A or with placebo. The abbreviation “ODU” stands for optical density unit. The abbreviation “anti-WHs” stands for antibodies against Woodchuck Hepatitis Virus Surface Antigen.

FIG. 3 shows serum Woodchuck Hepatitis Virus Surface Antigen concentration plotted against time (weeks) for Woodchucks chronically infected with WHV that were treated with either Compound A or with placebo. The abbreviation “ODU” stands for optical density unit. The abbreviation “WHsAg” stands for Woodchuck Hepatitis Virus Surface Antigen.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims.

On the contrary, the invention is intended to cover all alternatives, modifications, and equivalents, which may be included within the scope of the present invention as defined by the claims.

All documents referenced herein are each incorporated by reference in their entirety for all purposes.

### Definitions

Unless stated otherwise, the following terms and phrases as used herein are intended to have the following meanings. The fact that a particular term or phrase is not specifically defined should not be correlated to indefiniteness or lacking clarity, but rather terms herein are used within their ordinary meaning. When trade names are used herein, applicants intend to independently include the tradename product and the active pharmaceutical ingredient(s) of the tradename product.

As will be appreciated by those skilled in the art, Compound A is capable of existing in solvated or hydrated form. Such forms can be used in the practice of the present invention. Again, as will be appreciated by those skilled in the art, Compound A is capable of esterification. The scope of the present invention includes esters and other physiologically functional derivatives. The scope of the present invention also includes use of tautomeric forms, namely, tautomeric “enols” as herein described. In addition, the scope of the present invention includes use of prodrug forms of Compound A.

The term “prodrug” as used herein refers to any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically active compound.

“Ester” means any ester of a compound in which any of the —COOH functionality of the molecule is replaced by a —C(O)OR function, or in which any of the —OH functionality of the molecule are replaced with a —OC(O)R function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl, or substituted derivatives thereof. Esters can also include esters of “tautomeric enols”, for example, as shown below:

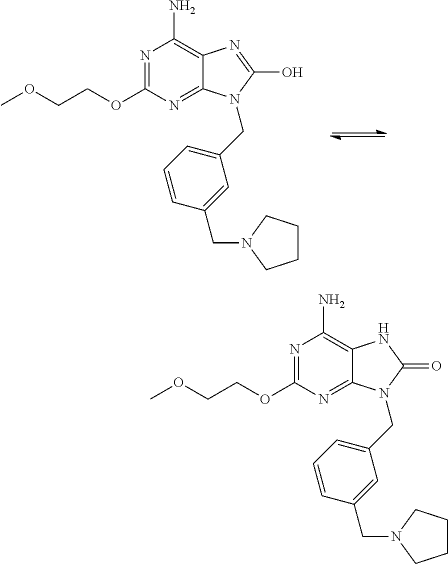

The term “ester” includes but is not limited to pharmaceutically acceptable esters thereof.

“Seroconversion” means the induction of a new immune response, or the enhancement of an existing immune response, against HBV in the body of a person infected with HBV, or against HCV in the body of a person infected with HCV, and a consequent reduction of the HBV viral load in the body of the person infected with HBV, or a consequent reduction of the HCV viral load in the body of the person infected with HCV. The immune response can include production of neutralizing antibodies against one or more HBV antigens or HCV antigens.

The term “immune response” refers to a response of the human immune system against HBV or HCV. For example, the immune response can be humoral (e.g., production of neutralizing antibodies against a surface antigen of HBV or HCV), cell-based (e.g., production of cytotoxic T lymphocytes), induction of antibody-dependent cellular cytotoxicity, or a combination of two or more of the foregoing types of immune responses.

As used herein, the term “treating a hepatitis B viral infection”, and grammatical equivalents thereof, means slowing or stopping the progression of a hepatitis B viral infection, or ameliorating at least one symptom of a hepatitis B viral infection, preferably ameliorating more than one symptom of a hepatitis B viral infection. For example, treatment of a hepatitis B virus infection can include reducing the HBV viral load in an HBV infected human being, and/or reducing the amount of inflammation present in the liver of an HBV infected human being, and/or reducing the severity of jaundice present in an HBV infected human being.

As used herein, the term “treating a hepatitis C viral infection”, and grammatical equivalents thereof, means slowing or stopping the progression of a hepatitis C viral infection, or ameliorating at least one symptom of a hepatitis C viral infection, preferably ameliorating more than one symptom of a hepatitis C viral infection. For example, treatment of a hepatitis C virus infection can include reducing the HCV viral load in an HCV infected human being, and/or reducing the amount of inflammation present in the liver of an HCV infected human being, and/or reducing the severity of jaundice present in an HCV infected human being.

A “therapeutically effective amount” of a drug substance, such as Compound A, is an amount of the drug substance that is effective to treat a hepatitis B viral infection, or a hepatitis C viral infection, in a human being.

As used herein, an “agonist” is a substance that stimulates its binding partner, typically a receptor.

As used herein, the term “viral load” refers to the concentration of a virus, such as HBV or HCV, in the blood of a mammal, such as a human being.

### Ether- and Ester-Forming Protecting Groups

Ester-forming groups include: (1) phosphonate ester-forming groups, such as phosphonamidate esters, phosphorothioate esters, phosphonate esters, and phosphon-bis-amidates; (2) carboxyl ester-forming groups, and (3) sulphur ester-forming groups, such as sulphonate, sulfate, and sulfinate.

### Compound A Used in the Present Invention

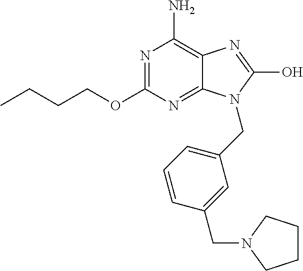

### 6-amino-2-butoxy-9-(3-(pyrrolidin-1-ylmethyl)benzyl)-9H-purin-8-ol

For ease of reference this compound, and its pharmaceutically acceptable salts, may be referred to herein as Compound A. Solvated forms or hydrated forms, salt forms, esterified forms, prodrugs, and tautomeric forms of Compound A may be used in the practice of the present invention.

### Pharmaceutical Formulations

Compound A may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference with regard to such formulation. Excipients include ascorbic acid and other antioxidants, chelating agents such as EDTA, carbohydrates such as dextrin, hydroxyalkylcellulose, hydroxyalkylmethylcellulose, stearin acid and the like. The pH of the formulations ranges from about 3 to about 11, but is ordinarily about 7 to 10.

While it is possible for an active ingredient to be administered alone it may be preferable to present them as pharmaceutical formulations. The formulations of the invention comprise at least one active ingredient, together with one or more acceptable carriers and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference with regard to such teaching. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

For administration to the eye or other external tissues e.g., mouth and skin, the formulations are preferably applied as a topical ointment or cream containing the active ingredient(s). When formulated in an ointment, the active ingredients may be employed with either a paraffinic or a water-miscible ointment base. Alternatively, the active ingredient(s) may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise Compound A, or a pharmaceutically acceptable salt thereof, together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

Compound A can also be formulated to provide controlled release of the active ingredient to allow less frequent dosing or to improve the pharmacokinetic or toxicity profile of the active ingredient. Accordingly, the invention also provided compositions comprising Compound A formulated for sustained or controlled release.

The effective dose of an active ingredient depends at least on the nature of the condition being treated, side effect profile, whether the compound is being used prophylactically (perhaps in lower doses) or against an active disease or condition, the method of delivery, and the pharmaceutical formulation, and can be determined by the clinician using conventional dose escalation studies.

The effective dose is in the range of from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 μg per day to about 30 mg per day, or such as from about 30 μg to about 300 μg per day.

It will be understood that the term “about” when used in connection with a dosage of Compound A (e.g., about 0.0001 mg/kg body weight per day) includes the value that is qualified by the term “about”. For example, the dosage range “from about 0.0001 mg/kg body weight per day to about 10 mg/kg body weight per day” includes the dosage range from 0.0001 mg/kg body weight per day to 10 mg/kg body weight per day.

### Routes of Administration

Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. For example, a severely ill patient may require intravenous administration of Compound A.

### Combination Therapy

Combinations of Compound A and one or more additional pharmaceutically active agent(s) may be used in the practice of the present invention to treat patients having an HBV or HCV infection. Useful active therapeutic agents for treating an HBV infection include reverse transcriptase inhibitors, such as lamivudine (Epivir®), adefovir (Hepsera®), tenofovir (Viread®), telbivudine (Tyzeka®), entecavir (Baraclude®), and Clevudine®. Other useful active therapeutic agents include immunomodulators, such as interferon alpha-2b (Intron A®), pegylated interferon alpha-2a (Pegasys®), interferon alpha 2a (Roferon®), interferon alpha N1, prednisone, predinisolone, Thymalfasin®, retinoic acid receptor agonists, 4-methylumbelliferone, Alamifovir®, Metacavir®, Albuferon®, agonists of TLRs (e.g., TLR-7 agonists), and cytokines.

Thus, in a further embodiment, the present application provides a combination pharmaceutical composition comprising:

a) Compound A or a pharmaceutically acceptable salt thereof; and

b) a second pharmaceutically active agent (or pharmaceutically acceptable salt thereof) selected from the group consisting of reverse transcriptase inhibitors (such as lamivudine (Epivir®), adefovir (Hepsera®), tenofovir (Viread®), telbivudine (Tyzeka®), entecavir (Baraclude®), and Clevudine®) and immunomodulators (such as interferon alpha-2b (Intron A®), pegylated interferon alpha-2a (Pegasys®), interferon alpha 2a (Roferon®), interferon alpha N1, prednisone, predinisolone, Thymalfasin®, retinoic acid receptor agonists, 4-methylumbelliferone, Alamifovir®, Metacavir®, Albuferon®, agonists of TLRs (e.g., TLR-7 agonists), and cytokines). The combination pharmaceutical composition may, for example, be in the form of a tablet, or, for example, in the form of a liquid suitable for injection into a patient, or, for example, in the form of a powder which can be dissolved for injection into a patient.

In yet another embodiment, the present application provides a method for treating an HBV infection, wherein the method comprises the step of co-administering, to a human being in need thereof, a therapeutically effective amount of Compound A and one or more additional active agents selected from the group consisting of reverse transcriptase inhibitors (such as lamivudine (Epivir®), adefovir (Hepsera®), tenofovir (Viread®), telbivudine (Tyzeka®), entecavir (Baraclude®), and Clevudine®) and immunomodulators (such as interferon alpha-2b (Intron A®), pegylated interferon alpha-2a (Pegasys®), interferon alpha 2a (Roferon®), interferon alpha N1, prednisone, predinisolone, Thymalfasin®, retinoic acid receptor agonists, 4-methylumbelliferone, Alamifovir®, Metacavir®, Albuferon®, agonists of TLRs (e.g., TLR-7 agonists), and cytokines). In the practice of this aspect of the invention, typically the amounts of Compound A and the one or more additional therapeutic agent(s) are individually therapeutic, but it is within the scope of the invention for the amounts of Compound A and the one or more additional therapeutic agent(s) to be subtherapeutic by themselves, but the combination of Compound A and the one or more additional therapeutic agent(s) is therapeutic.

With regard to treatment for HCV, other active therapeutic ingredients or agents include interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, HCV IRES inhibitors, pharmacokinetic enhancers, and other drugs for treating HCV, or mixtures thereof.

More specifically, other active therapeutic ingredients or agents for treating HCV include:

(1) interferons selected from the group consisting of pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha-2b XL, BLX-883 (Locteron), DA-3021, glycosylated interferon alpha-2b (AVI-005), PEG-Infergen, PEGylated interferon lambda-1 (PEGylated IL-29), belerofon, and mixtures thereof;

(2) ribavirin and its analogs selected from the group consisting of ribavirin (Rebetol, Copegus), taribavirin (Viramidine), and mixtures thereof;

(3) HCV NS3 protease inhibitors selected from the group consisting of boceprevir (SCH-503034 , SCH-7), telaprevir (VX-950), TMC435350, BI-1335, BI-1230, MK-7009, VBY-376, VX-500, BMS-790052, BMS-605339, PHX-1766, AS-101, YH-5258, YH5530, YH5531, ITMN-191, and mixtures thereof;

(4) alpha-glucosidase 1 inhibitors selected from the group consisting of celgosivir (MX-3253), Miglitol, UT-231B, and mixtures thereof;

(5) hepatoprotectants selected from the group consisting of IDN-6556, ME 3738, silibilin, MitoQ, and mixtures thereof;

(6) nucleoside or nucleotide inhibitors of HCV NS5B polymerase selected from the group consisting of R1626, R7128 (R4048), IDX184, IDX-102, BCX-4678, valopicitabine (NM-283), MK-0608, and mixtures thereof;

(7) non-nucleoside inhibitors of HCV NS5B polymerase selected from the group consisting of PF-868554, VCH-759, VCH-916, JTK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, GS-9190, and mixtures thereof;

(8) HCV NS5A inhibitors selected from the group consisting of AZD-2836 (A-831), A-689, and mixtures thereof;

(9) TLR-7 agonists selected from the group consisting of ANA-975, SM-360320, and mixtures thereof;

(10) cyclophillin inhibitors selected from the group consisting of DEBIO-025, SCY-635, NIM811, and mixtures thereof;

(11) HCV IRES inhibitors selected from the group consisting of MCI-067,

(12) pharmacokinetic enhancers selected from the group consisting of BAS-100, SPI-452, PF-4194477, TMC-41629, roxythromycin, and mixtures thereof; and

(13) other drugs for treating HCV selected from the group consisting of thymosin alpha 1 (Zadaxin), nitazoxanide (Alinea, NTZ), BIVN-401 (virostat), PYN-17 (altirex), KPE02003002, actilon (CPG-10101), KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, VX-497 (merimepodib), and mixtures thereof.

Thus, in a further embodiment, the present invention provides a combination pharmaceutical composition comprising:

a) Compound A or a pharmaceutically acceptable salt thereof; and

b) a second pharmaceutically active agent (or pharmaceutically acceptable salt thereof) effective to treat HCV.

In yet another embodiment, the present application provides a method for treating an HCV infection, wherein the method comprises the step of co-administering, to a human being in need thereof, a therapeutically effective amount of Compound A and one or more of the additional active agents described herein that are effective to treat HCV.

In the practice of this aspect of the invention, typically the amounts of Compound A and the one or more additional therapeutic agent(s) are individually therapeutic, but it is within the scope of the invention for the amounts of Compound A and the one or more additional therapeutic agent(s) to be subtherapeutic by themselves, but the combination of Compound A and the one or more additional therapeutic agent(s) is therapeutic.

Co-administration of Compound A with one or more other active agents generally refers to simultaneous or sequential administration of Compound A and one or more other active agents, such that Compound A and one or more other active agents are both present in the body of the patient. Simultaneous administration of Compound A and one or more additional therapeutic agents can be achieved, for example, by mixing the Compound A and one or more additional therapeutic agents in a single dosage form, such as a tablet or injectable solution. Again by way of example, simultaneous administration of Compound A and one or more additional therapeutic agents can be achieved by co-packaging, for example in a blister pack, Compound A and at least one other therapeutic agent, so that a patient can remove and consume individual doses of Compound A and the other therapeutic agent.

Co-administration includes administration of unit dosages of Compound A before or after administration of unit dosages of one or more other active agents, for example, administration of Compound A within seconds, minutes, or hours of the administration of one or more other active agents. For example, a unit dose of Compound A can be administered first, followed within seconds or minutes by administration of a unit dose of one or more other active agents. Alternatively, a unit dose of one or more other active agents can be administered first, followed by administration of a unit dose of Compound A within seconds or minutes. In some cases, it may be desirable to administer a unit dose of Compound A first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of Compound A.

In still yet another embodiment, the present application provides for the use of Compound A, or a pharmaceutically acceptable salt thereof, for the preparation of a medicament for treating an HBV infection or an HCV infection.

### Methods of Treatment

As will be appreciated by those skilled in the art, when treating a viral infection such as HBV or HCV, such treatment may be characterized in a variety of ways and measured by a variety of endpoints. The scope of the present invention is intended to encompass all such characterizations.

In one aspect the present invention provides methods for treating a hepatitis

B viral infection or a hepatitis C viral infection, wherein each of the methods includes the step of administering to a human subject infected with hepatitis B virus or hepatitis C virus a therapeutically effective amount of Compound A or a pharmaceutically acceptable salt thereof. Typically, the human subject is suffering from a chronic hepatitis B infection or a chronic hepatitis C infection, although it is within the scope of the present invention to treat people who are acutely infected with HBV or HCV.

Treatment in accordance with the present invention typically results in the stimulation of an immune response against HBV or HCV in a human being infected with HBV or HCV, respectively, and a consequent reduction in the viral load of HBV or HCV in the infected person. Examples of immune responses include production of antibodies (e.g., IgG antibodies) and/or production of cytokines, such as interferons, that modulate the activity of the immune system. The immune system response can be a newly induced response, or can be boosting of an existing immune response. Treatment in accordance with the present invention can cause seroconversion against one or more HBV antigens or HCV antigens.

The viral load can be determined by measuring the amount of HBV DNA or HCV DNA present in the blood. For example, blood serum HBV DNA can be quantified using the Roche COBAS Amplicor Monitor PCR assay (version 2.0; lower limit of quantification, 300 copies/mL [57 IU/mL]) and the Quantiplex bDNA assay (lower limit of quantification, 0.7 MEq/mL; Bayer Diagnostics, formerly Chiron Diagnostics, Emeryville, Calif.). The amount of antibodies against specific HBV or HCV antigens (e.g., hepatitis B surface antigen (HBsAG)) can be measured using such art-recognized techniques as enzyme-linked immunoassays and enzyme-linked immunoabsorbent assays. For example, the amount of antibodies against specific HBV or HCV antigens can be measured using the Abbott AxSYM microparticle enzyme immunoassay system (Abbott Laboratories, North Chicago, Ill.).

Compound A can be administered by any useful route and means, such as by oral or parenteral (e.g., intravenous) administration. Therapeutically effective amounts of Compound A are from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 μg to about 30 mg per day, or such as from about 30 μg per day to about 300 μg per day.

The frequency of dosage of Compound A will be determined by the needs of the individual patient and can be, for example, once per day or twice, or more times, per day. Administration of Compound A continues for as long as necessary to treat the HBV or HCV infection. For example, Compound A can be administered to a human being infected with HBV or HCV for a period of from 7 days to 180 days or, for example, for a period of from 20 days to 90 days or, for example, for a period of from 30 days to 60 days.

Administration can be intermittent, with a period of several or more days during which a patient receives a daily dose of Compound A, followed by a period of several or more days during which a patient does not receive a daily dose of Compound A. For example, a patient can receive a dose of Compound A every other day, or three times per week. Again by way of example, a patient can receive a dose of Compound A each day for a period of from 1 to 14 days, followed by a period of 7 to 21 days during which the patient does not receive a dose of Compound A, followed by a subsequent period (e.g., from 1 to 14 days) during which the patient again receives a daily dose of Compound A. Alternating periods of administration of Compound A, followed by non-administration of Compound A, can be repeated as clinically required to treat the patient.

As described more fully supra, Compound A can be administered with one or more additional therapeutic agent(s) to a human being infected with HBV or HCV. The additional therapeutic agent(s) can be administered to the infected human being at the same time as Compound A, or before or after administration of Compound A.

In another aspect, the present invention provides a method for ameliorating a symptom associated with an HBV infection or HCV infection, wherein the method comprises administering to a human subject infected with hepatitis B virus or hepatitis C virus a therapeutically effective amount of Compound A, or a pharmaceutically acceptable salt thereof, wherein the therapeutically effective amount is sufficient to ameliorate a symptom associated with the HBV infection or HCV infection. Such symptoms include the presence of HBV virus particles (or HCV virus particles) in the blood, liver inflammation, jaundice, muscle aches, weakness and tiredness.

In a further aspect, the present invention provides a method for reducing the rate of progression of a hepatitis B viral infection, or a hepatitis C virus infection, in a human being, wherein the method comprises administering to a human subject infected with hepatitis B virus or hepatitis C virus a therapeutically effective amount of Compound A, or a pharmaceutically acceptable salt thereof, wherein the therapeutically effective amount is sufficient to reduce the rate of progression of the hepatitis B viral infection or hepatitis C viral infection. The rate of progression of the infection can be followed by measuring the amount of HBV virus particles or HCV virus particles in the blood.

In another aspect, the present invention provides a method for reducing the viral load associated with HBV infection or HCV infection, wherein the method comprises administering to a human being infected with HBV or HCV a therapeutically effective amount of Compound A, or a pharmaceutically acceptable salt thereof, wherein the therapeutically effective amount is sufficient to reduce the HBV viral load or the HCV viral load in the human being.

In a further aspect, the present invention provides a method of inducing or boosting an immune response against Hepatitis B virus or Hepatitis C virus in a human being, wherein the method comprises administering a therapeutically effective amount of Compound A, or a pharmaceutically acceptable salt thereof, to the human being, wherein a new immune response against Hepatitis B virus or Hepatitis C virus is induced in the human being, or a preexisting immune response against Hepatitis B virus or Hepatitis C virus is boosted in the human being. Seroconversion against HBV or HCV can be induced in the human being. Examples of immune responses include production of antibodies, such as IgG antibody molecules, and/or production of cytokine molecules that modulate the activity of one or more components of the human immune system.

Induction of seroconversion against HCV or HBV in patients chronically infected with either of these viruses is an unexpected property of Compound A. In clinical practice, an HBV patient, or HCV patient, is treated with Compound A, alone or in combination with one or more other therapeutic agents, until an immune response against HBV or HCV is induced or enhanced and the viral load of HBV or HCV is reduced. Thereafter, although the HBV or HCV virus may persist in a latent form in the patient's body, treatment with Compound A can be stopped, and the patient's own immune system is capable of suppressing further viral replication. In patients treated in accordance with the present invention and who are already receiving treatment with an antiviral agent that suppresses replication of the HBV virus or HCV virus, there may be little or no detectable viral particles in the body of the patient during treatment with the antiviral agent(s). In these patients, seroconversion will be evident when the antiviral agent(s) is no longer administered to the patient and there is no increase in the viral load of HBV or HCV.

In the practice of the present invention, an immune response is induced against one or more antigens of HBV or HCV. For example, an immune response can be induced against the HBV surface antigen (HBsAg), or against the small form of the HBV surface antigen (small S antigen), or against the medium form of the HBV surface antigen (medium S antigen), or against a combination thereof. Again by way of example, an immune response can be induced against the HBV surface antigen (HBsAg) and also against other HBV-derived antigens, such as the core polymerase or x-protein.

Induction of an immune response against HCV or HBV can be assessed using any technique that is known by those of skill in the art for determining whether an immune response has occurred. Suitable methods of detecting an immune response for the present invention include, among others, detecting a decrease in viral load in a subject's serum, such as by measuring the amount of HBV DNA or HCV DNA in a subject's blood using a PCR assay, and/or by measuring the amount of anti-HBV antibodies, or anti-HCV antibodies, in the subject's blood using a method such as an ELISA.

### EXAMPLE 1

### Synthesis of Compound A

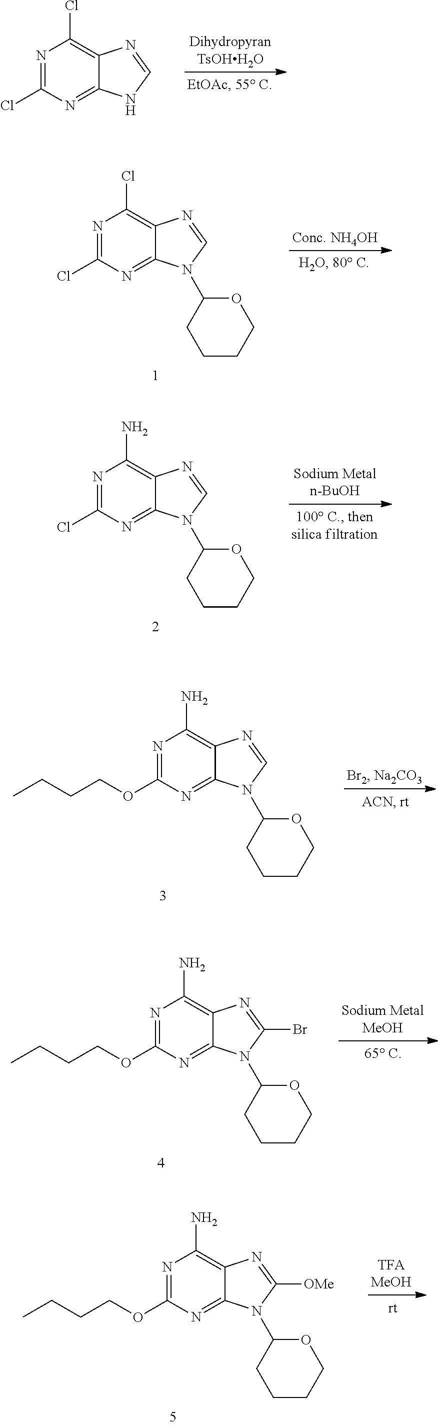

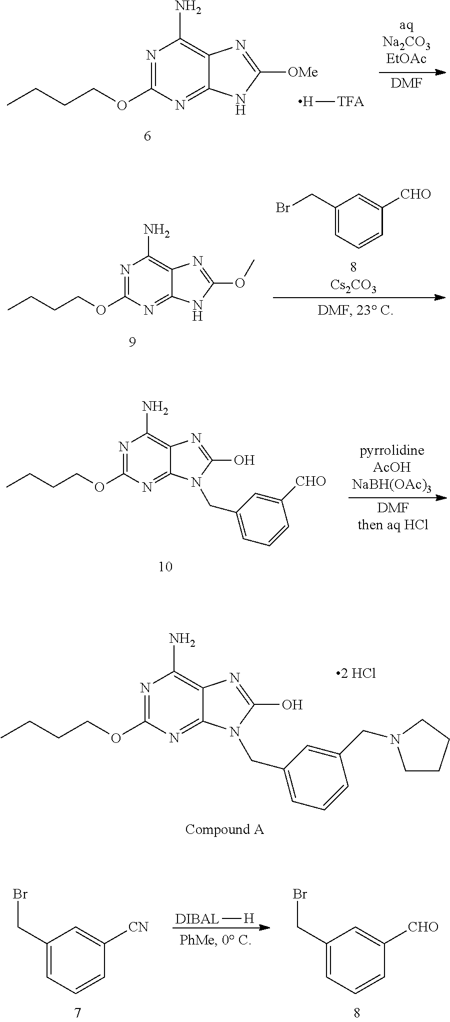

### Compound 1

To a solution of TsOH—H₂O (505 mg) in EtOAc (750 ml) in a water bath, was gradually added 2,6-dichloropurine (49.75 g). The temperature was then raised to 50° C. To this solution, 3,4-dihydro-2H-pyran (31.5 mL) was added dropwise at ˜1.0 ml/min over 30 min through a slow addition pressure equalizing funnel. After addition was done, the reaction was stirred for another 30 min, and then allowed to cool to ambient temperature. The reaction was quenched with ice-cold water (˜300 mL) and then 3% w/v aq. NH₃ was added (10 mL) to the rapidly stirred mixture. The organic phase was collected, washed with ice-cold water (2×250 ml), brine (100mL), then dried (Na₂SO₄), filtered, and concentrated down. The residue was diluted with heptane (500 mL) and heated to 100° C. in a water bath with a reflux condenser and the top of condenser open to air. When most of the material was dissolved, the flask was allowed to cool in a water bath to ambient temperature overnight. Crystals were collected and dried in a vacuum oven giving 59.72 g (83%) product 1. ¹H-NMR (300 MHz, CDCl₃) d: 5.75 (app. d, J=10.4 Hz, 1H), 4.17 (app. d, J=9.7 Hz, 1H), 3.77 (app. t, J=11.3 Hz, 1H), 2.22-1.88 (m, 3H), 1.87-1.62 (m, 3H). LCMS-ESI: (m/z): [M+H⁺] calc'd for C₁₀H₁₀Cl₂N₄O, 272.0; found, 272.6 (M+H⁺, ³⁵Cl, ³⁵Cl), 274.6 (M+2+H⁺, ³⁷Cl, ³⁵_{Cl),} 276.7 (M+4+H⁺, ³⁷Cl, ³⁷Cl)

### Compound 2

A 2 L flask was charged with compound 1 (56.81 g), concentrated NH₄OH (500 ml) and de-ionized water (250 mL). Reaction was fitted with a condenser, and the top of the condenser was vented to the back of the fume hood. Reaction was carefully heated at 80° C. for 24 h. LC/MS at this time showed <10% bis-amination, hence the reaction was immediately cooled down, filtered and the filter cake was concentrated from acetonitrile (200 mL) followed by dichloromethane (2×200 ml). The resulting fluffy yellow powder was dried in a vacuum oven to give 50.88 g product (97%). ¹H-NMR (300 MHz, CDCl₃) d: 7.99 (s, 1H), 6.42 (s, broad, 2H), 5.68 (app. d, J=9.7 Hz, 1H), 4.14 (app. d, J=10.4 Hz, 1H), 3.76 (app. t, J=9.4 Hz, 1H), 2.16-1.56 (m, 6H). LCMS-ESI: (m/z): [M+H⁺] calc'd for C₁₀H₁₂ClN₅O, 253.1; found, 253.8 (M+H⁺, ³⁵Cl), 255.8 (M+2+H⁺, ³⁷Cl)

### Compound 3

A solution of NaO-n-Bu in n-BuOH was made by adding Na (15 g) portion wise (˜500 mg per piece) to n-BuOH (600 mL) under N₂. The reaction was heated at 100° C. until all sodium had dissolved. Then to the solution was carefully added compound 2 (50.8 g) and the reaction mixture was reacted at 100° C. for 6 h. The reaction mixture was then cooled to room temperature and H₂O (250 ml) was added. Organic phase was collected and the aqueous phase was extracted with Et₂O (2×200 mL). Then organic layers were combined, washed with brine (200 mL), dried (Na₂SO₄) and concentrated down. The residue oil was diluted with EtOAc (100 mL) and filtered through a 7-inch silica plug with the aid of EtOAc to remove impurities. The resulting solution was concentrated down and then stripped from Et₂O/Hexane (1:1, 500 ml) at 40° C. to give compound 3 as a yellow powder (49.48 g, 84%). ¹H-NMR (300 MHz, CDCl₃) d: 7.84 (s, 1H), 5.72 (s, broad, 2H), 5.62 (dd, J=10.0, 2.5 Hz, 1H), 4.30 (t, J=6.5 Hz, 2H), 4.13 (app. d, J=11.3 Hz, 1H), 3.74 (app td, J=11.3, 2.6 Hz, 1H), 2.11-1.90 (m, 3H), 1.82-1.56 (m, 5H), 1.49 (qt, J=7.4, 7.4 Hz, 2H), 0.96 (t, J=7.4 Hz, 3H). LCMS-ESI: (m/z): [M+H⁺] calc'd for C₁₄H₂₁N₅O₂, 291.2; found, 291.9 (M+H⁺).

### Compound 4

A solution of Br₂ (27.5 g) in acetonitrile (170 mL) was treated with Na₂CO₃ (27 g) for 10 min at room temperature. The supernatant solution (150 mL) was decanted into an addition funnel, then gradually added to a mixture of compound 3 (15.0 g), Na₂CO₃ (30 g) and ACN (300 mL) at 5 mL/min at room temperature. The reaction was monitored by HPLC. After 4 hrs, the reaction was quenched with 10% w/v aq Na₂SO₃ (300 mL). The organic phase was collected after 10 min, dried (Na₂SO₄), filtered, concentrated down (including any residual water), stripped from MeOH and triturated with Hexane. The solid was collected to give 17.75 g product (93%). ¹H-NMR (300 MHz, CDCl₃) d: 5.60 (app. d, J=11.0 Hz, 1H), 5.48 (s, broad, 2H), 4.30 (app. s, broad, 2H), 4.16 (app. d, broad, J=9.3 Hz, 1H), 3.70 (app. t, broad, J=11.3 Hz, 1H), 3.10-2.92 (m, 1H), 2.17-2.00 (m, 1H), 1.90-1.40 (m, 8H), 0.96 (app. s, broad, 3H). LCMS-ESI: (m/z): [M+H⁺] calc'd for C₁₄H₂₀BrN₅O, 369.1; found, 369.8 (M+H⁺, ⁷⁹Br), 371.8 (M+2+H⁺, ⁸¹Br).

### Compound 5

A solution of NaOMe in MeOH was made by adding sodium metal (˜3 g) portion wise to MeOH (500 mL) at room temperature. Once all the sodium had reacted, compound 4 (17.5 g) was added and the reaction mixture was heated to 65° C. under N₂, and the reaction was monitored by HPLC. When the reaction was finished, it was concentrated down to ˜20 mL total volume, then water (150 mL) was added, followed by EtOAc (100 mL). The organic phase was collected and the aqueous phase was extracted by EtOAc (2×75 mL). The organic phase was combined and dried (Na₂SO₄). Then it was filtered and concentrated down to ˜50 mL and filtered through a 2-inch plug of silica, eluting with EtOAc. The filtrate was concentrated down to give 12.3 g product as an orange powder (81%). ¹H-NMR (300 MHz, CDCl₃) d: 5.47 (dd, J=11.3, 2.4 Hz, 1 H), 5.42 (s, broad, 2H), 4.26 (t, J=6.6 Hz, 2H), 4.09 (app. d, J=6.7 Hz, 1H), 4.08 (s, 3H), 3.66 (app. t, J=11.6 Hz, 1H), 2.74 (app. ddd, J=24.7, 12.2, 3.9 Hz, 1H), 2.00 (app. d, J=12.5 Hz, 1H), 1.78-1.50 (m, 6H), 1.46 (qt, J=7.4, 7.4 Hz, 2H), 0.93 (t, J=7.4 Hz, 3H). LCMS-ESI: (m/z): [M+H⁺] calc'd for C₁₅H₂₃N₅O₃, 321.2; found, 321.9 (M+H⁺).

### Compound 6

A mixture of compound 5 (12.3 g) and MeOH (100 mL) was carefully treated with TFA (10.0 mL) at room temperature. Then the reaction mixture was heated to 60° C. for 5 hrs under N₂. Reaction was then concentrated to give the product as a TFA salt. ¹H-NMR (300 MHz, DMSO-d₆) d: 7.92 (s, broad, 2H), 4.32 (t, J=7.4 Hz, 2H), 4.05 (s, 3H), 1.70 (tt, J=7.4, 7.4 Hz, 2H), 1.41 (qt, J=7.4, 7.4 Hz, 2H), 0.93 (t, J=7.4 Hz, 3H). ¹⁹F NMR (282 MHz, DMSO-d₆) d: −74.9 (s, 3F). LCMS-ESI: (m/z): [M+H⁺] calc'd for C₁₀H₁₅N₅O₂ (CF₃COOH absent), 237.1; found, 238.0 (M+H⁺).

### Compound 8

A 2 L round-bottom was charged with 3-(bromomethyl)-benzonitrile 7 (48.0 g) and dry toluene (480 mL). After cooling to 0° C., DIBAL-H (1.0 M in PhMe, 306 mL) was added over a 90 min period via pressure-equalizing addition funnel under N₂. Once addition was complete, the reaction was allowed to stir an additional 90 min. Then 1.0 M aq HCl (1.2 L) was added carefully, and the reaction was allowed to stir for 15 min. The organic phase was collected, and the aq phase was extracted (2×250 mL Et₂O). All organic extracts were combined, dried (MgSO₄), filtered, and concentrated. The oily residue was purified on a 330 g normal phase silica gel column (combiflash, EtOAc/Hex 10:90 to 50:50, gradient), giving the 3-(bromomethyl)-benzaldehyde (37.14 g, 77% yield). ¹H-NMR (300 MHz, CDCl₃) d: 9.98 (s, 1H), 7.86 (app. s, 1H), 7.77 (app.d, J=7.6 Hz, 1H), 7.63 (app. d, J=7.7 Hz, 1H), 7.48 (app. dd, J=7.7, 7.6 Hz, 1H), 4.51 (s, 2H). LCMS-ESI failed to provide reliable data.

### Compound 9

To a homogeneous solution of the TFA salt 6 (10.22 g) in DMF (20 mL) was added EtOAc (30 mL) at 23° C. To this was added a 10% w/v solution of aq Na₂CO₃ (30 mL). Immediately, a flocculent grey precipitate formed as the reaction was vigorously stirred. After stirring for 5 min, the reaction was filtered over coarse glass frits (40-60 grade porosity). The filter cake was dried in a vacuum oven at 45° C. overnight, giving the free base 9 as a grey free-flowing powder. Mass: 6.69 g, yield: 97%. The solid was analyzed via LCMS (C18 gemini column, aq AcOH/CH₃CN 95:5-0:100) and found to be ˜90% pure. Desired product has a retention time of 1.65 minutes during a 3.5 min run. ¹H-NMR (300 MHz, DMSO-d₆) d: 6.62 (s, broad, 2H), 4.12 (t, J=7.4 Hz, 2H), 3.98 (s, 3H), 1.63 (tt, J=7.4, 7.4 Hz, 2H), 1.38 (qt, J=7.4, 7.4 Hz, 2H), 0.91 (t, J=7.4 Hz, 3H). LCMS-ESI: (m/z): [M+H⁺] calc'd for C₁₀H₁₅N₅O₂, 237.1; found, 238.0 (M+H⁺).

### Compound 10

A 250 mL r.b. flask was charged with free base 9 (5.00 g, 1.00 equiv), 3-(bromomethyl)-benzaldehyde (8) (4.16 g, 1.00 equiv), and Cs₂CO₃ (Aldrich, 27.4 g, 4.00 equiv). DMF (100 mL) was added, and the reaction was stirred vigorously at 23° C. During the first 4 h, the reaction reached complete conversion as determined by LCMS; complete consumption of free base 6 was seen. 3 new peaks showed up in the LCMS in a 1:1:2 ratio, having identical m/z ratios (C18 gemini column, aq AcOH/CH₃CN 95:5-0:100, 3.5 min run. T₁=1.79 min, T₂=1.99 min, and T₃=2.20 min 1:1:2). The peak at 2.20 min is the desired product. By this time, the reaction had become green, and stirring was continued for an additional 20 h. At this time, LCMS analysis via the method above revealed a mixture that was primarily the T₃ compound (>10:1 vs all other isomers). The reaction was then filtered over medium-porosity glass frits (10-15 grade porosity); the cake being washed with DMF (20 mL) to retrieve intracted product from the Cs₂CO₃. No aqueous work-up was done, instead the reaction was directly loaded to a Sep-Pak C-18 reversed-phase column, (loading 10 mL/86 g column packing); eluent: 0.05% w/v aq HCl/CH₃CN (95/5-0/100). The product-containing fractions were tested for purity via LCMS, and those that were 85% or greater were combined, concentrated in vacuo at 65° C., giving the aldehyde 10 as an orange solid, which had also undergone hydrolysis of the O-8 methyl group. Mass: 3.62 g, yield: 50%. ¹H-NMR (300 MHz, DMSO-d₆) d: 10.68 (s, broad, 1H), 9.98 (s, 1H), 7.86-7.80 (m, 1H), 7.82 (app. s, 1H), 7.69-7.54 (m, 2H), 5.23 (s, broad, 2H), 4.98 (s, 2H), 4.22 (t, J=7.4 Hz, 2H), 1.63 (tt, J=7.4, 7.4 Hz, 2H), 1.36 (qt, J=7.4, 7.4 Hz, 2H), 0.88 (t, J=7.4 Hz, 3H). LCMS-ESI C18 gemini column, eluent: aq AcOH/CH₃CN 95:5-0:100, 3.5 min run. (T_{R}=2.15 min), (m/z): [M+H⁺] calc'd for C₁₇H₁₉N₅O₃, 341.2; found, 342.0 (M+H⁺).

### Compound A.

A solution of aldehyde 10 (3.5 g) in DMF (15 mL) was treated sequentially with glacial AcOH (3.5 mL), pyrrolidine (3.5 mL), and NaBH(OAc)₃ (3.5 g) at 23° C. The reaction was stirred overnight and monitored via LCMS. Once complete conversion was achieved, the reaction was quenched with 1.0 M aq HCl (15 mL). The entire reaction was directly loaded onto a Sep-Pak C-18 reversed-phase column, (loading 30 mL/330 g column packing); eluent: 0.05% w/v aq HCl/CH₃CN (95/5-0/100). The product-containing fractions were tested for purity via LCMS, and those that were 90% or greater were combined, concentrated in vacuo at 65° C., then lyophilized from aq HCl/CH₃CN giving Compound A as the dihydrochloride salt. Mass: 2.70 g, yield: 56%. ¹H-NMR (300 MHz, DMSO-d₆) d: 10.32 (s, broad, 1H), 10.13 (s, broad, 1H), 7.49-7.33 (m, 4H), 6.71 (s, broad, 1H), 4.89 (s, 2H), 4.32 (app. d, J=6.7 Hz, 2H), 4.14 (t, J=7.4 Hz, 2H), 3.38-3.26 (m, 2H), 3.10-2.97 (m, 2H), 2.05-1.92 (m, 2H), 1.91-1.77 (m, 2H), 1.61 (tt, J=7.4, 7.4 Hz, 2H), 1.36 (qt, J=7.4, 7.4 Hz, 2H), 0.90 (t, J=7.4 Hz, 3H). LCMS-ESI C18 gemini column, eluent: aq AcOH/CH₃CN 95:5-0:100, 3.5 min run. (T_{R}=1.60 min), (m/z): [M+H⁺] calc'd for C₁₇H₁₉N₅O₃ (2 HCl molecules absent), 396.2; found, 397.2 (M+H⁺).

### EXAMPLE 2

### Induction of Cytokines by Compound A in Human Peripheral Blood Mononuclear Cells (PBMCs) in Vitro

Frozen human PBMCs were thawed and used to seed the wells of 96 well plates (7.5×10⁵cells/well in 190 μL/well culture medium (CM)). The cells were incubated for 1 hour at 37° C. at a 5% CO₂. Ten microliters of CM containing Compound A was added to each well except for the control wells which received 10 μL CM that did not contain Compound A. The concentrations of Compound A that were tested were 0.1 μM, 0.01 μM, and 0.001 μM. The plates were incubated at 37° C., 5% CO₂, for 24 hours, then centrifuged at 1200 rpm for 10 minutes. After centrifugation the supernatant was collected and stored at −80° C. The supernatant was assayed using Luminex and Upstate multi-plex kits for the presence of various cytokines.

The concentrations of the cytokines, in pg/mL, are shown in Table 1. Compound A stimulated the production of a range of cytokines in human PBMCs in vitro.

TABLE 1

| Compound | | | | | | | | IL- | | |
|---|---|---|---|---|---|---|---|---|---|---|
| A [μM] | IFN-α | IFN-β | IFN-γ | TNF-α | IL-6 | IL1-α | IL1-β | 1ra | IL-10 | IL-12 |
| 0.1 | 80 | 97 | 5400 | 3665 | 93554 | 280 | 2836 | 4934 | 1523 | 773 |
| 0.01 | 122 | 170 | 4578 | 1449 | 66284 | 94 | 752 | 4530 | 992 | 386 |
| 0.001 | 208 | 324 | 2099 | 82 | 27664 | 7 | 138 | 3216 | 735 | 61 |
| zero | 8 | 10 | 39 | 82 | 7444 | 7 | 1048 | 99 | 40 | 71 |

### EXAMPLE 3

### Induction of Interferon Alpha by Compound A in Cynomolqus Monkeys

A single dose of Compound A was administered orally to cynomolgus monkeys (3 or more animals per dose group) and serum was collected at 4 hours and 8 hours after dosing. Serum samples were analyzed for levels of interferon-alpha by ELISA. Prior to dosing, serum interferon-alpha levels were near or below the level of detection in each animal. The limit of quantitation (LOQ) for IFN-a based on cynomolgus monkey IFN-a standard was 625 pg/mL. Table 2 shows the average interferon alpha levels induced by Compound A.

TABLE 2

| | Average peak serum |
|---|---|
| Compound A Dose | Interferon-alpha levels (pg/ml) |
| (mg/kg) | (Mean ± standard deviation) |
| 2.5 | 772923 ± 281529 |
| 0.05 | 42741 ± 29223 |
| 0.02 | 26148 ± 13873 |
| 0.005 | 7829 ± 9192 |
| Predose | 719 ± 871 |

Additionally, multiple doses of Compound A were administered to Cynomolgus monkeys, and the concentrations of interferon alpha were measured.

Compound A was dosed orally once per day to Cynomolgus monkeys (3 animals per dose group), and serum was collected on Days 1, 7 and 15 at 4 and 8 hours after dosing. Serum samples were analyzed for levels of interferon-alpha by ELISA. Prior to dosing on Day 1, serum interferon-alpha levels were below the level of detection in each animal. The data from this study are shown in Table 3. Interferon-alpha was induced by Compound A.

TABLE 3

| | |
|---|---|
| Oral Dose of | Average peak serum Interferon-alpha |
| Compound A | levels (pg/ml) on designated day |

| (mg/kg) | Predose | Day 1 | Day 7 | Day 15 |
|---|---|---|---|---|
| 0.005 | BLQ | 3233 | 6709 | 24053 |
| 0.05 | BLQ | 87570 | 12175 | 42648 |

BLQ: below the limit of detection for the assay; <625 pg/ml

### EXAMPLE 4

### Induction of Cytokines by Compound A in Mice

Compound A was dosed once per day for 14 days by oral gavage, at 0.5 mg/kg or 2.5 mg/kg, in CD-1 mice. Mouse serum samples were collected on day 1 and day 14, and serum cytokine levels were determined using the following method. The assay for interferon-a was done by ELISA (VeriKine™ Mouse Interferon Alpha (Mu-IFN-a) ELISA Kit, Product Number: 42100-1, PBL Biomedical Laboratories, New Brunswick, N.J.) and the other serum cytokines were assayed with Luminex and Milliplex bead kits. Cytokine levels were determined using a nonlinear five point parameter curve for interpolation of data using the fit=(A+((B−A)/(1+(((B−E)/(E−A))*((x/C)̂D))))). The results are shown in Table 4 where the cytokine concentrations are expressed in units of pg/mL.

TABLE 4

| Oral Dose | | | | | | |
|---|---|---|---|---|---|---|
| of | Average Serum Cytokine Levels on Day 1 ± Standard Deviation at 2 | | | | | |
| Compound | hours Post-dose in Mice. | | | | | |
| A (mg/kg) | IL6 | IL-10 | IL1-β | IL12p40 | IFN-a | TNF-a |
| 0.5 | 257.2 ± 420.9 | 3.2 ± 0 | 4.8 ± 2.7 | 3.2 ± 0 | 296.3 ± 322.4 | 41.7 ± 42.4 |
| 2.5 | 6710.2 ± 3657.4 | 143.4 ± 102.7 | 20.8 ± 16.1 | 10 ± 11.7 | 1091.8 ± 413.6 | 407.1 ± 258.1 |

### EXAMPLE 5

### Induction of Cytokines by Compound A in Healthy Woodchucks

Compound A was administered orally to adult, WHV-negative woodchucks at two different doses. Three male woodchucks received Compound A at 0.05 mg/kg and three other male woodchucks at 0.5 mg/kg. Whole blood samples (4 mls) were collected from each woodchuck prior to dosing at T0, and then at 4, 8, 12, and 24 hours post-dose using EDTA-containing collection tubes.

The induction of an immune response in woodchucks following administration of Compound A was determined by measuring the mRNA expression of cytokines and interferon-inducible genes in whole blood samples collected at different time points. Total RNA was isolated using the QIAamp RNA Blood Mini Kit (Qiagen) according to the manufacturer's specifications. RNA was eluted into 40 μl nuclease-free water and stored at −70° C. The concentration of RNA was determined spectrophotometrically at OD 260 nm. Two μg of RNA were treated with DNase I (Invitrogen) and reverse transcribed to cDNA with MultiScribe Reverse Transcriptase (Applied Biosystems) using random hexamers. Triplicates of 2 μl cDNA were amplified by real time PCR on an ABI PRISM 7000 Sequence Detection instrument (Applied Biosystems) using SYBR GREEN Master Mix (Applied Biosystems) and woodchuck-specific primers. Amplified target genes included IFN-α, IFN-γ, TNF-α, IL-2, IL-6, IL-10 IL-12, 2′5′-OAS, IDO, and MxA. Woodchuck β-actin mRNA expression was used to normalize target gene expression. Transcription levels of woodchuck cytokines and interferon-inducible genes were represented by the formula 2ΔCt, where ΔCt indicates the difference in the threshold cycle between β-actin and target gene expression. Results were further represented as a fold-change from the transcription level at T0.

The results are shown in Tables 5A through 5T.

TABLE 5A
IFN-a mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 3.28 | 2.43 | 1.0 | 0.0 |
| 4 | 7.21 | 1.28 | 3.1 | 2.0 |
| 8 | 12.99 | 8.75 | 4.2 | 1.9 |
| 12 | 3.82 | 1.97 | 1.3 | 0.4 |
| 24 | 2.63 | 0.45 | 1.0 | 0.9 |

TABLE 5B
IFN-a mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 4.81 | 1.12 | 1.0 | 0.0 |
| 4 | 19.14 | 4.96 | 3.9 | 0.2 |
| 8 | 48.85 | 15.33 | 10.4 | 3.8 |
| 12 | 7.12 | 2.13 | 1.5 | 0.2 |
| 24 | 4.15 | 1.07 | 0.9 | 0.1 |

TABLE 5C
IFN-γ mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 1.28 | 0.33 | 1.0 | 0.0 |
| 4 | 2.03 | 0.63 | 1.6 | 0.2 |
| 8 | 4.34 | 1.34 | 3.4 | 0.3 |
| 12 | 2.64 | 0.96 | 2.0 | 0.3 |
| 24 | 1.82 | 0.38 | 1.2 | 0.3 |

TABLE 5D
IFN-γ mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 1.20 | 0.38 | 1.0 | 0.0 |
| 4 | 2.80 | 0.30 | 2.6 | 1.3 |
| 8 | 7.81 | 2.26 | 6.7 | 1.3 |
| 12 | 4.36 | 0.94 | 3.8 | 0.7 |
| 24 | 2.16 | 0.94 | 1.9 | 0.1 |

TABLE 5E
TNF-a mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 6.82 | 0.65 | 1.0 | 0.0 |
| 4 | 23.83 | 1.65 | 3.5 | 0.3 |
| 8 | 20.72 | 2.20 | 3.0 | 0.1 |
| 12 | 35.05 | 7.99 | 5.2 | 1.3 |
| 24 | 20.53 | 2.54 | 3.2 | 0.6 |

TABLE 5F
TNF-a mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 6.87 | 0.68 | 1.0 | 0.0 |
| 4 | 43.18 | 14.04 | 6.2 | 1.5 |
| 8 | 31.19 | 8.94 | 4.5 | 1.0 |
| 12 | 62.30 | 26.41 | 9.0 | 3.2 |
| 24 | 30.71 | 2.71 | 4.5 | 1.0 |

TABLE 5G
IL-2 mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 1.14 | 0.03 | 1.0 | 0.0 |
| 4 | 2.11 | 0.28 | 1.9 | 0.3 |
| 8 | 1.74 | 0.70 | 1.5 | 0.6 |
| 12 | 2.43 | 0.42 | 2.1 | 0.3 |
| 24 | 3.64 | 0.17 | 3.2 | 0.1 |

TABLE 5H
IL-2 mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 1.79 | 1.59 | 1.0 | 0.0 |
| 4 | 4.32 | 3.96 | 2.2 | 0.3 |
| 8 | 3.12 | 2.09 | 2.0 | 1.5 |
| 12 | NC | NC | NC | NC |
| 24 | 13.38 | 14.09 | 5.9 | 0.8 |

NC: not calculated due to insufficient data

TABLE 5I
IL-6 mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 4.15 | 0.32 | 1.0 | 0.0 |
| 4 | 9.75 | 2.07 | 2.3 | 0.5 |
| 8 | 10.57 | 2.10 | 2.6 | 0.7 |
| 12 | 6.22 | 0.76 | 1.5 | 0.3 |
| 24 | 4.30 | 0.73 | 1.1 | 0.2 |

TABLE 5J
IL-6 mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 4.40 | 1.47 | 1.0 | 0.0 |
| 4 | 21.84 | 13.00 | 4.6 | 1.5 |
| 8 | 18.57 | 0.49 | 4.6 | 1.6 |
| 12 | 6.25 | 3.04 | 1.4 | 0.3 |
| 24 | 4.75 | 1.38 | 1.2 | 0.2 |

TABLE 5K
IL-10 mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 2.47 | 0.26 | 1.0 | 0.0 |
| 4 | 4.14 | 0.81 | 1.7 | 0.3 |
| 8 | 8.48 | 1.90 | 3.4 | 0.4 |
| 12 | 7.35 | 1.00 | 3.0 | 0.3 |
| 24 | 6.08 | 0.04 | 2.3 | 0.1 |

TABLE 5L
IL-10 mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 2.63 | 0.84 | 1.0 | 0.0 |
| 4 | 4.01 | 1.18 | 1.5 | 0.1 |
| 8 | 17.31 | 6.68 | 6.6 | 1.6 |
| 12 | 14.92 | 7.11 | 5.6 | 1.4 |
| 24 | 8.74 | 3.67 | 3.3 | 0.1 |

TABLE 5M
IL-12 mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 1.64 | 0.41 | 1.0 | 0.0 |
| 4 | 7.27 | 0.90 | 4.5 | 0.6 |
| 8 | 7.35 | 0.68 | 4.7 | 1.4 |
| 12 | 21.12 | 7.60 | 12.7 | 1.9 |
| 24 | NC | NC | NC | NC |

NC: not calculated due to insufficient data

TABLE 5N
IL-12 mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 1.64 | 0.38 | 1.0 | 0.0 |
| 4 | 5.74 | 1.67 | 3.8 | 1.8 |
| 8 | 6.86 | 1.44 | 4.2 | 0.7 |
| 12 | 49.93 | 19.04 | 30.1 | 5.9 |
| 24 | 11.55 | 4.13 | 7.0 | 0.2 |

TABLE 5O
2′5′-OAS mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 3.55 | 0.75 | 1.0 | 0.0 |
| 4 | 13.89 | 5.40 | 3.8 | 0.7 |
| 8 | 16.96 | 11.78 | 4.8 | 3.6 |
| 12 | 54.92 | 12.28 | 15.6 | 3.0 |
| 24 | 36.91 | 0.96 | 11.9 | 1.2 |

TABLE 5P
2′5′-OAS mRNA expression in peripheral blood of woodchucks
following oral dosing with Compound A at 0.5 mg/kg

| | | | Fold- | |
|---|---|---|---|---|
| Time | | | change | |
| (hrs) | Mean | SD | from T₀ | SD |
| 0 | 4.92 | 0.76 | 1.0 | 0.0 |
| 4 | 33.05 | 34.28 | 6.5 | 6.3 |
| 8 | 41.13 | 21.21 | 8.3 | 3.7 |
| 12 | 884.87 | 281.57 | 185.6 | 75.5 |
| 24 | 64.27 | 24.67 | 14.4 | 8.2 |

TABLE 5Q
IDO mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 1.31 | 0.73 | 1.0 | 0.0 |
| 4 | 1.30 | 0.59 | 1.1 | 0.3 |
| 8 | 2.05 | 0.16 | 2.2 | 1.6 |
| 12 | 10.64 | 9.29 | 7.7 | 3.3 |
| 24 | 26.71 | 17.17 | 25.6 | 6.9 |

TABLE 5R
IDO mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 2.26 | 0.80 | 1.0 | 0.0 |
| 4 | 22.67 | 19.93 | 9.1 | 5.8 |
| 8 | 30.62 | 24.96 | 11.6 | 8.9 |
| 12 | 89.01 | 55.80 | 37.0 | 16.5 |
| 24 | 1217.20 | 401.15 | 454.2 | 179.8 |

TABLE 5S
MxA mRNA expression in peripheral blood of woodchucks
following oral dosing with Compound A at 0.05 mg/kg.

| | | | Fold- | |
|---|---|---|---|---|
| Time | | | change | |
| (hrs) | Mean | SD | from T₀ | SD |
| 0 | 1.56 | 0.32 | 1.0 | 0.0 |
| 4 | 2.26 | 1.05 | 1.4 | 0.4 |
| 8 | 5.93 | 3.43 | 3.8 | 1.8 |
| 12 | 18.70 | 5.62 | 12.5 | 5.7 |
| 24 | 117.33 | 1.15 | 69.3 | 11.2 |

TABLE 5T
MxA mRNA expression in peripheral blood of woodchucks
following oral dosing with Compound A at 0.5 mg/kg.

| | | | Fold- | |
|---|---|---|---|---|
| Time | | | change | |
| (hrs) | Mean | SD | from T₀ | SD |
| 0 | 1.56 | 0.41 | 1.0 | 0.0 |
| 4 | 5.96 | 3.38 | 3.6 | 1.1 |
| 8 | 12.86 | 8.98 | 7.9 | 4.0 |
| 12 | 71.77 | 26.33 | 45.3 | 5.8 |
| 24 | 758.52 | 131.93 | 518.8 | 265.8 |

The foregoing data shows that Compound A induced dose-dependent increases in the mRNA expression of interferon (IFN)-α, IFN-γ, tumor necrosis factor (TNF)-alpha, interleukin (IL)-2, IL-6, IL-10, IL-12, 2′5′-oligoadenylate synthetase (2′5′-OAS), indoleamine 2′3′-dioxygenase (IDO), and interferon-induced cellular resistance mediator protein (MxA).

mRNA expression of IFN-α, IFN-γ, IL-6, and IL-10 was maximal at 8 hours post-dose. mRNA expression of TNF-α, IL-12, and 2′5′-OAS was maximal at 12 hours post-dose. mRNA expression of IL-2, IDO, and MxA was maximal at 24 hours post-dose.

### EXAMPLE 6

### Seroconversion in Woodchucks Chronically Infected with Woodchuck Hepatitis Virus (WHV)

Compound A or placebo was administered orally to five woodchucks per group that were chronic carriers of woodchuck hepatitis virus (WHV). Compound A was administered at a dose of 0.5 mg/kg/day for 28 days. Blood samples were collected prior to dosing and multiple times during and after the 28 day dosing period. Antiviral activity of Compound A was assessed by comparing the serum WHV DNA of treated WHV carrier woodchucks with control WHV carrier woodchucks receiving vehicle. The ability of Compound A to cause seroconversion in chronically infected animals was assessed by comparing the serum antibody levels against the woodchuck hepatitis virus surface antigen (anti-WHsAg) in infected animals to the anti-WHsAg antibody levels in placebo treated animals.

The woodchucks used in this study were born to WHV-negative females and reared in environmentally controlled laboratory animal facilities. Woodchucks were inoculated at 3 days of age with 5 million woodchuck infectious doses of a standardized WHV inoculum (cWHV7P1 or WHV7P2). Woodchucks selected for use developed WHV surface antigen (WHsAg) serum antigenemia and became chronic WHV carriers. The chronic carrier status of these woodchucks was confirmed prior to initiation of drug treatment.

Serum WHV DNA concentrations were measured before treatment, during treatment, and during the post-treatment follow-up period at frequent intervals. WHV viremia in serum samples was assessed by dot blot hybridization using three replicate volumes (10 μl) of undiluted serum (sensitivity, 1.0×10⁷ WHV genome equivalents per ml [WHVge/ml]) compared with a standard dilution series of WHV recombinant DNA plasmid (pWHV8).

Levels of Woodchuck Hepatitis Virus surface antigen (WHsAg) and antibodies to WHsAg (anti-WHs) were determined before treatment, during treatment, and during the post-treatment follow-up period at frequent intervals, using WHV-specific enzyme immunoassays.

Antiviral activity of Compound A was assessed by comparing the serum WHV DNA and the hepatic WHV nucleic acids of treated WHV carrier woodchucks with control WHV carrier woodchucks receiving vehicle.

Immune stimulatory activity of Compound A to cause seroconversion was assessed by comparing the serum levels of WHsAg and antibodies to WHsAg (anti-WHsAg).

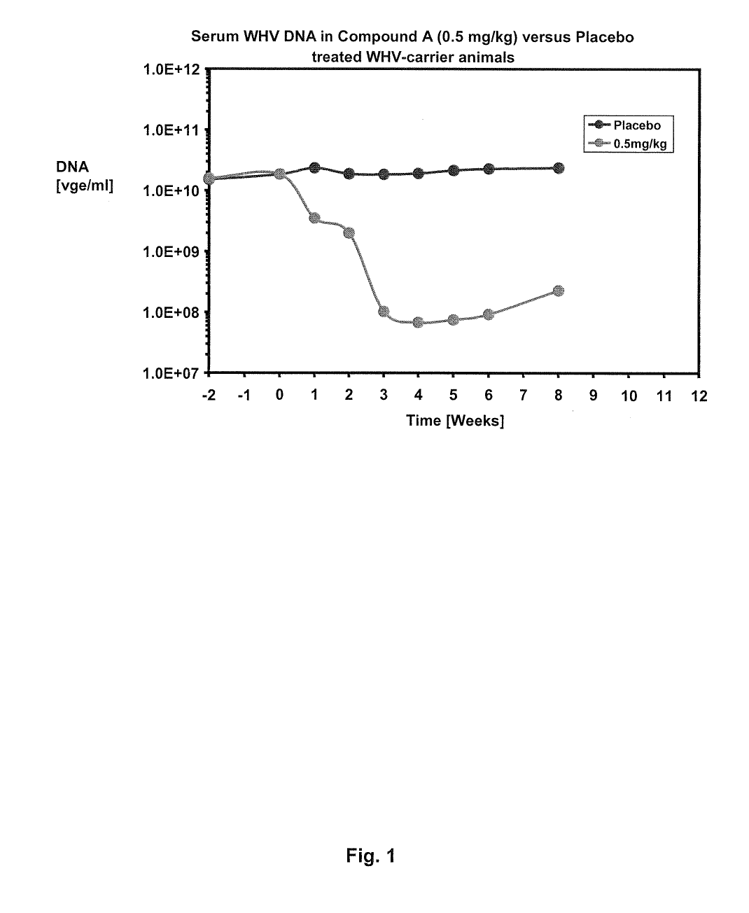

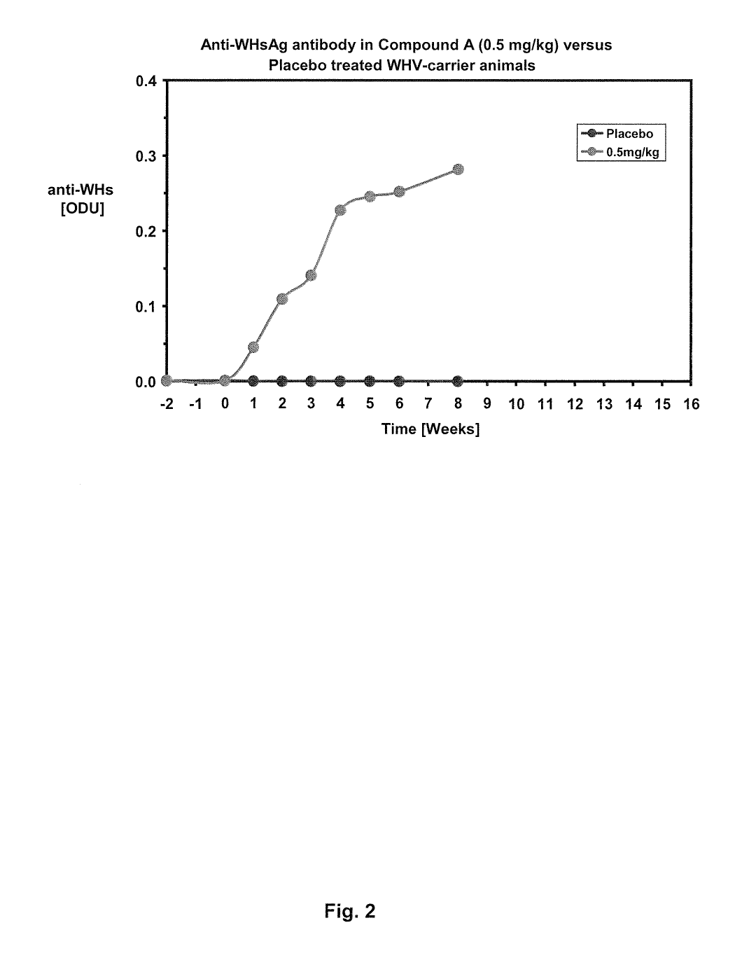

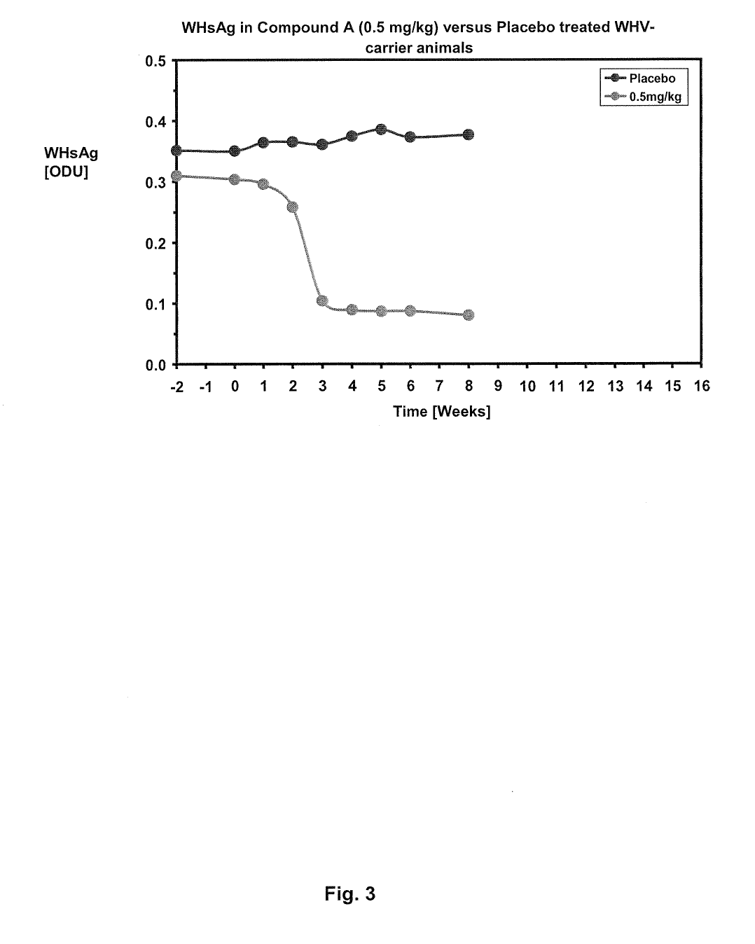

The data in FIG. 1 shows that treatment of Woodchucks with Compound A caused a substantial reduction in serum WHV DNA concentrations. The data in FIG. 2 shows that treatment of Woodchucks with Compound A stimulated production of antibodies against Woodchuck Hepatitis Virus Surface Antigen (WHsAg). The data in FIG. 3 shows that treatment of Woodchucks with Compound A caused a substantial reduction in serum levels of WHsAg. Taken together the data in FIGS. 1-3 show that Compound A induced seroconversion against WHV in woodchucks and a consequent reduction in HBV viral load.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed:

1. A method for treating a hepatitis B viral infection comprising administering to a human subject infected with hepatitis B virus a therapeutically effective amount of 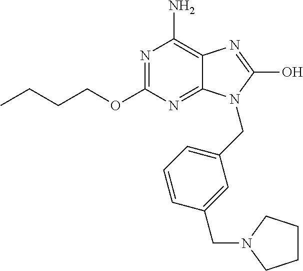
or a pharmaceutically acceptable salt thereof.

2. The method of claim 1 wherein the human subject is chronically infected with hepatitis B virus.

3. The method of claim 1 wherein the human subject lacks any detectable HBV virus after treatment with 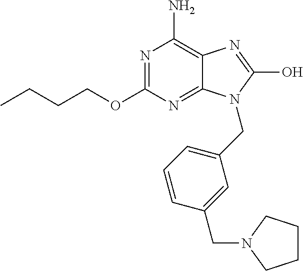
or a pharmaceutically acceptable salt thereof.

4. The method of claim 1 wherein the infected human subject has a viral load of HBV, and the viral load is reduced after treatment of the human subject with 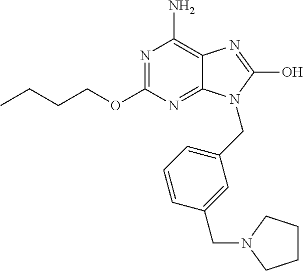
or a pharmaceutically acceptable salt thereof.

5. The method of claim 1 wherein 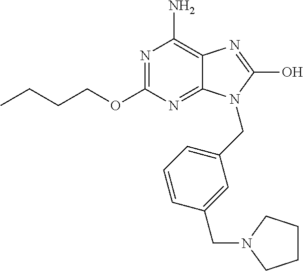
or a pharmaceutically acceptable salt thereof, is orally administered to the human subject.

6. The method of claim 1 wherein 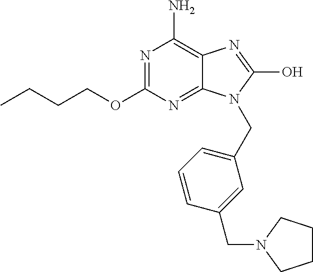
or a pharmaceutically acceptable salt thereof, is parenterally administered to the human subject.

7. The method of claim 1 wherein the therapeutically effective amount is from about 0.00001 mg/kg body weight per day to about 10 mg/kg body weight per day.

8. The method of claim 1 wherein the therapeutically effective amount is from about 0.001 mg/kg body weight per day to about 1 mg/kg body weight per day.

9. The method of claim 1 wherein the therapeutically effective amount is from about 0.01 mg/kg body weight per day to about 1 mg/kg body weight per day.

10. The method of claim 1 wherein the therapeutically effective amount is from about 0.05 mg/kg body weight per day to about 0.5 mg/kg body weight per day.

11. The method of claim 1 wherein the therapeutically effective amount is administered to the human being once a day for a period of from 7 days to 180 days.

12. The method of claim 11 wherein the therapeutically effective amount is administered to the human being once a day for a period of from 20 days to 90 days.

13. The method of claim 11 wherein the therapeutically effective amount is administered to the human being once a day for a period of from 30 days to 60 days.

14. The method of claim 1 wherein 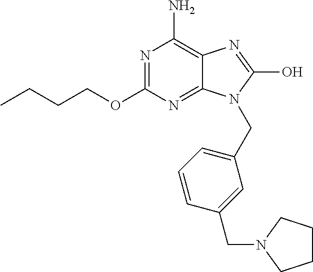
or a pharmaceutically acceptable salt thereof, is administered to the human being more than once per day.

15. The method of claim 1 further comprising administering an additional therapeutic agent to the human being.

16. The method of claim 15 wherein the additional therapeutic agent is selected from the group consisting of lamivudine, adefovir, tenofovir, telbivudine, entecavir, interferon alpha-2b, pegylated interferon alpha-2a, interferon alpha 2a, interferon alpha N1, prednisone, predinisolone, Thymalfasin®, retinoic acid receptor agonists, 4-methylumbelliferone, Alamifovir®, Metacavir®, Albuferon®, cytokines and agonists of toll-like receptors.

17. The method of claim 15 wherein the additional therapeutic agent is administered to the human being at the same time as 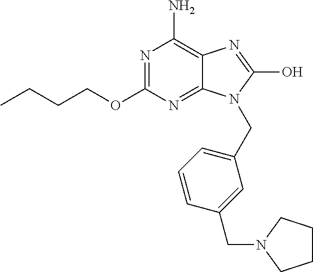
or a pharmaceutically acceptable salt thereof.

18. The method of claim 15 wherein the additional therapeutic agent is administered to the human being before or after administration of 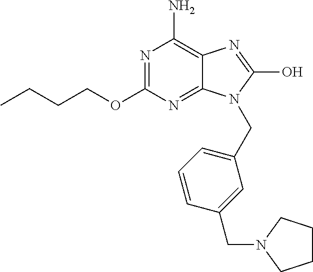
or a pharmaceutically acceptable salt thereof, to the human being.

19. The method of claim 1 wherein an immune response against Hepatitis B Virus is induced or boosted in the human being, or seroconversion with respect to Hepatitis B Virus is induced in the human being, by the therapeutically effective amount of 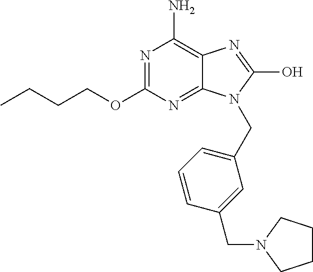
or a pharmaceutically acceptable salt thereof.

20. The method of claim 19 wherein an immune response against Hepatitis B Virus is induced in the human being.

21. The method of claim 19 wherein an immune response against Hepatitis B Virus is boosted in the human being.

22. The method of claim 19 wherein seroconversion against Hepatitis B Virus is induced in the human being.

23. The method of claim 19 wherein the immune response comprises production of antibodies or the production of cytokines that modulate the activity of the immune system.

24. A method for treating a hepatitis C viral infection comprising administering to a human subject infected with hepatitis C virus a therapeutically effective amount of 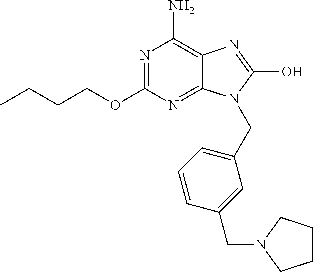
or a pharmaceutically acceptable salt thereof.

25. The method of claim 24 wherein the human subject is chronically infected with hepatitis C virus.

26. The method of claim 24 wherein the human subject lacks any detectable HCV virus after treatment with 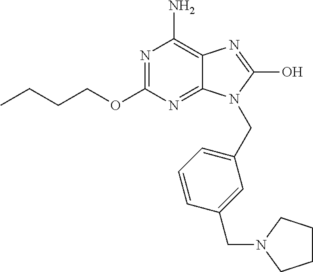
or a pharmaceutically acceptable salt thereof.

27. The method of claim 24 wherein the infected human subject has a viral load of HCV, and the viral load is reduced after treatment of the human subject with 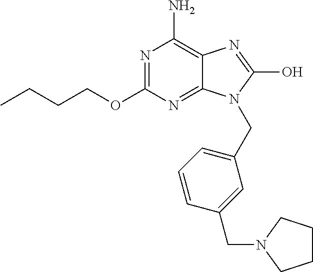
or a pharmaceutically acceptable salt thereof.

28. The method of claim 24 wherein 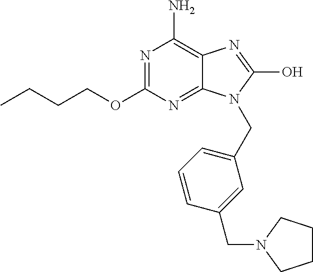
or a pharmaceutically acceptable salt thereof, is orally administered to the human subject.

29. The method of claim 24 wherein 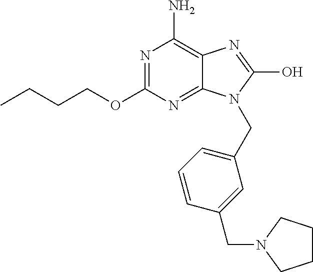
or a pharmaceutically acceptable salt thereof, is parenterally administered to the human subject.

30. The method of claim 24 wherein the therapeutically effective amount is from about 0.00001 mg/kg body weight per day to about 10 mg/kg body weight per day.

31. The method of claim 24 wherein the therapeutically effective amount is from about 0.001 mg/kg body weight per day to about 1 mg/kg body weight per day.

32. The method of claim 24 wherein the therapeutically effective amount is from about 0.01 mg/kg body weight per day to about 1 mg/kg body weight per day.

33. The method of claim 24 wherein the therapeutically effective amount is from about 0.05 mg/kg body weight per day to about 0.5 mg/kg body weight per day.

34. The method of claim 24 wherein the therapeutically effective amount is administered to the human being once a day for a period of from 7 days to 180 days.

35. The method of claim 34 wherein the therapeutically effective amount is administered to the human being once a day for a period of from 20 days to 90 days.

36. The method of claim 34 wherein the therapeutically effective amount is administered to the human being once a day for a period of from 30 days to 60 days.

37. The method of claim 24 wherein 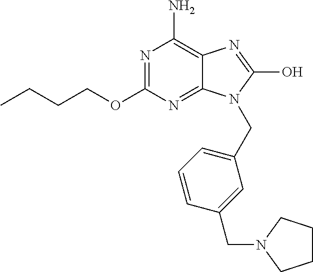
or a pharmaceutically acceptable salt thereof, is administered to the human being more than once per day.

38. The method of claim 24 further comprising administering an additional therapeutic agent to the human being.

39. The method of claim 38 wherein the additional therapeutic agent is selected from the group consisting of interferons, ribavirin or its analogs, HCV NS3 protease inhibitors, alpha-glucosidase 1 inhibitors, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, and HCV IRES inhibitors.

40. The method of claim 38 wherein the additional therapeutic agent is administered to the human being at the same time as 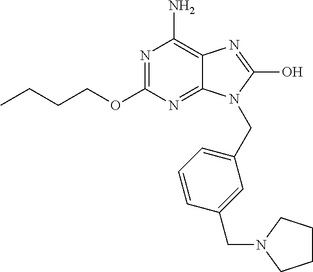
or a pharmaceutically acceptable salt thereof.

41. The method of claim 38 wherein the additional therapeutic agent is administered to the human being before or after administration of 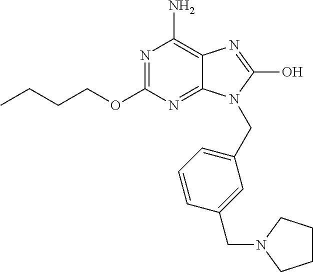
or a pharmaceutically acceptable salt thereof, to the human being.

42. The method of claim 24 wherein an immune response against Hepatitis C Virus is induced or boosted in the human being, or seroconversion with respect to Hepatitis C Virus is induced in the human being, by the therapeutically effective amount of 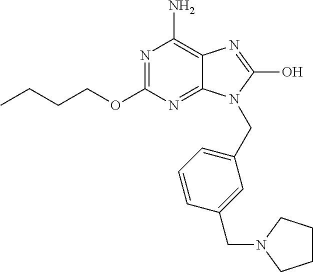
or a pharmaceutically acceptable salt thereof.

43. The method of claim 42 wherein an immune response against Hepatitis C Virus is induced in the human being.

44. The method of claim 42 wherein an immune response against Hepatitis C Virus is boosted in the human being.

45. The method of claim 42 wherein seroconversion against Hepatitis C Virus is induced in the human being.

46. The method of claim 42 wherein the immune response comprises production of antibodies or the production of cytokines that modulate the activity of the immune system.

47. The method of claim 1 or claim 24 wherein 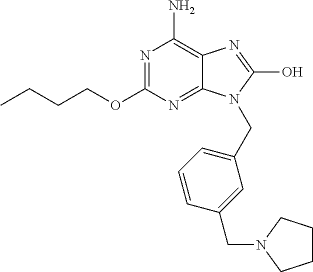
or a pharmaceutically acceptable salt thereof is intermittently administered to the human subject.

48. The method of claim 1 or claim 24 wherein the therapeutically effective amount is from about 0.3 μg per day to about 30 mg per day.

49. A method for ameliorating at least one symptom of HBV or HCV infection in a human being, wherein the method comprises the steps of (a) testing a human subject to determine if the human subject is infected with HCV or HBV, and (b) if the human subject is infected with HBV or HCV then administering to the human subject an amount of Compound A that is sufficient to ameliorate at least one symptom of the HCV or HBV infection.
